(19) United States
(12) Patent Application Publication
(10) Pub. No.: US 2017/0246442 A1
(43) Pub. Date: Aug. 31, 2017

(54) ORGANIZER ANCHOR ASSEMBLY FOR DIALYSIS SYSTEM

(71) Applicant: Fresenius Medical Care Holdings, Inc., Waltham, MA (US)

(72) Inventors: John A. Biewer, Waltham, MA (US); Alan Wandesforde, Lafayette, CA (US)

(21) Appl. No.: 15/437,812

(22) Filed: Feb. 21, 2017

Related U.S. Application Data
(60) Provisional application No. 62/299,847, filed on Feb. 25, 2016.

(51) Int. Cl. A61M 39/08; A61M 1/28

(52) U.S. Cl. CPC A61M 39/08; A61M 1/28; A61M 2209/084; A61M 2205/505

(57) ABSTRACT

An organizer anchor assembly may include an organizer unit coupled to an anchor base having a flat bottom with sufficient surface area in contact with a table or other surface to secure the organizer assembly in place during use. The organizer unit holds and organizes lines of a medical device, such as a peritoneal dialysis machine. The anchor base may be flat to contact the cart or table surface and/or may include a perpendicular portion designed to wrap around an edge of the cart or table surface. The organizer anchor assembly may be beneficially used in connection with a home dialysis machine in which lines and connectors are desirably organized and anchored during a dialysis treatment. Portions of the organizer anchor assembly may be made of polymer materials that provide a frictional interface in contact with a surface to resist movement of the organizer anchor assembly across the surface.

### RELATED APPLICATIONS

This application claims priority to US Prov. App. 62/299,847 filed Feb. 25, 2016, which is incorporated herein by reference.

### TECHNICAL FIELD

This application is related to the field of dialysis systems.

### BACKGROUND

Dialysis is a treatment used to support a patient with insufficient renal function. The two principal dialysis methods are hemodialysis and peritoneal dialysis.

During hemodialysis (HD), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water, in the blood. In this way, the dialysis machine acts as an artificial kidney for cleansing the blood.

During peritoneal dialysis (PD), the patient's peritoneal cavity is periodically infused with dialysate. The membranous lining of the patient's peritoneum acts as a natural semi-permeable membrane that allows diffusion and osmosis exchanges to take place between the solution and the blood stream. These exchanges across the patient's peritoneum result in the removal of waste products, including solutes like urea and creatinine, from the blood, and regulate the levels of other substances, such as sodium and water, in the blood.

HD treatments are typically performed on a patient multiple times a week using an HD machine in a clinic or home environment with each treatment lasting a least a few hours. PD treatments are done several times a day on a patient, often at home and often performed overnight while a patient is asleep using an automated PD machine called a PD cycler. A PD cycler is designed to control the entire PD process so that it can be performed at home without clinical staff in attendance. Many PD cyclers are designed to automatically infuse, dwell, and drain dialysate to and from the patient's peritoneal cavity. The treatment typically lasts for several hours, often beginning with an initial drain cycle to empty the peritoneal cavity of used or spent dialysate. The sequence then proceeds through the succession of fill, dwell, and drain phases that follow one after the other. Each phase is called a cycle. Dialysis machines are typically equipped with interfaces for receiving inputs and providing information to users during treatments.

Dialysis machines may have a disposable set which has several connectors and often many feet of tubing (also referred to as “lines”) used in connection with the dialysis treatment and through which medical fluid flows during the dialysis treatment. It is important that the connectors not touch dirty surfaces and become contaminated, which may lead to an infection such as peritonitis.

An organizer unit, such as a stay*safe organizer produced by Fresenius Medical Care, may be used to hold patient connectors to support aseptic techniques and one handed operation for making and unmaking the connection of the patient to the disposable sets for PD treatments, including automated peritoneal dialysis (APD) and continuous ambulatory peritoneal dialysis (CAPD) treatments. The organizer may be mounted on or near the PD cycler to position it in a good position for the patient to use (e.g. facing towards the patient, close to the patient to allow easy access, short distance to the catheter exit site, good visibility). The organizer unit may further include control components used to control flows of medical fluids during a therapy treatment. The organizer may include suction cup feet, but the use of suction cup feet may suffer problems securing the organizer to a surface.

Accordingly, it would be desirable to provide an improved assembly for anchoring an organizer used to hole patient connectors during a dialysis treatment.

### SUMMARY

According to the system described herein, an organizer anchor assembly includes an organizer unit that is configured to receive and secure a line of a medical device; and an anchor base coupled to the organizer unit. The anchor base includes a contact portion comprising a polymer material that, when in contact with a surface, resists movement of the organizer anchor assembly across the surface. The medical device may be a dialysis machine, such as a peritoneal dialysis machine. The contact portion may be an impact-resistant polymer material layer of the anchor base, and the polymer material may be silicone. The contact portion of the anchor base may cover at least 50% of a surface area of a side of the organizer anchor assembly that contacts the surface, and/or the contact portion of the anchor base may extend substantially along an entire length of the organizer unit. The contact portion of the anchor base may further include a portion that is substantially perpendicular to a plane of the anchor base that is in contact with the cart or table surface.

According further to the system described herein, a medical system comprises a medical device having lines through which medical fluid flows during a medical treatment and an organizer anchor assembly. The organizer anchor assembly includes an organizer unit that is configured to receive and secure a line of a medical device; and an anchor base coupled to the organizer unit. The anchor base includes a contact portion comprising a polymer material that, when in contact with a surface, resists movement of the organizer anchor assembly across the surface. The medical device may be a dialysis machine, such as a peritoneal dialysis machine.

The contact portion may be an impact-resistant polymer material layer of the anchor base, and the polymer material may be silicone. The contact portion of the anchor base may cover at least 50% of a surface area of a side of the organizer anchor assembly that contacts the surface, and/or the contact portion of the anchor base may extend substantially along an entire length of the organizer unit. The contact portion of the anchor base may further include a portion that is substantially perpendicular to a plane of the anchor base that is in contact with the cart or table surface.

### BRIEF DESCRIPTION OF THE DRAWINGS

Embodiments of the system described herein will be described in further detail below using the drawings, which are briefly described as follows. FIG. 1 shows a schematic illustration of a dialysis system using an organizer anchor assembly according to an embodiment of the system described herein.

FIG. 2 is a schematic view of the organizer anchor assembly according to an embodiment of the system described herein.

FIG. 3 is a schematic view showing an underside of the organizer anchor assembly, specifically the side of an anchor base' that would physically contact the cart or table surface.

FIG. 4 is a schematic view of an embodiment for an organizer anchor assembly in which an anchor base is shown having a portion that extends substantially perpendicular to the underside of an organizer unit.

FIG. 5 is a schematic view of a dialysis system including an organizer anchor assembly that may be removably secured to a table surface at a desired location on a table surface and receive lines from a dialysis machine.

### DETAILED DESCRIPTION

FIG. 1 shows a schematic illustration of a dialysis system using an organizer anchor assembly according to an embodiment of the system described herein. The dialysis system may be a peritoneal dialysis (PD) system 100 that includes a PD machine or cycler 102 seated on a cart 104. Although a particular PD system is principally discussed herein, it is noted that the system described herein may be used in connection with various configurations and types of PD machines, including PD machines having cassettes, pumps (e.g., pneumatic based pumps and/or peristaltic pumps), gravity-driven cyclers, and/or with other types of dialysis systems and/or other medical devices having connectors and medical tubing or lines through which flows medical fluid. For example, the system described herein may also be used in connection with a home hemodialysis (HD) system.

The PD cycler 102 includes a housing 106, a door 108, and a cassette interface 110 that contacts a disposable PD cassette 112 when the cassette 112 is disposed within a cassette compartment formed between the cassette interface 110 and the closed door 108. A heater tray 116 is positioned on top of the housing 106. The heater tray 116 is sized and shaped to accommodate a bag of dialysate (e.g. a 5 liter bag of dialysate). The PD cycler 102 also includes a user interface such as a touch screen display 118 and additional control buttons 120 that can be operated by a user (e.g. a caregiver or a patient) to allow, for example, set up, initiation, and/or termination of a PD treatment.

As illustrated, dialysate bags 122 are suspended from support fingers on the sides of the cart 104, and a heater bag 124 is positioned in the heater tray 116. In other embodiments, the system described herein may be used in connection with dialysate supplied from bags positioned at other locations and/or via other mechanisms and devices for preparing and delivering dialysate to/from the dialysate machine and a patient. The dialysate bags 122 and the heater bag 124 are connected to the cassette 112 via dialysate bag lines 126 and a heater bag line 128, respectively. The dialysate bag lines 126 can be used to pass dialysate from dialysate bags 122 to the cassette 112 during use, and the heater bag line 128 can be used to pass dialysate back and forth between the cassette 112 and the heater bag 124 during use. In addition, a patient line 130 and a drain line 132 are connected to the cassette 112. The patient line 130 can be connected to a patient's abdomen via a catheter and can be used to pass dialysate back and forth between the cassette 112 and the patient's peritoneal cavity during use. The drain line 132 can be connected to a drain or drain receptacle and can be used to pass dialysate from the cassette 112 to the drain or drain receptacle during use. As further discussed elsewhere herein, one or more of the lines 126, 128, 130, 132 may be organized and/or secured using an embodiment of the system described herein.

The PD cycler 102 includes a control unit 140 (e.g. a processor) and one or more interface components or sensors 151, 152, such as a microphone/speaker assembly and/or a motion sensor that detects motion of a nearby user. The control unit 140 can receive signals from and transmit signals to the touch screen display 118, the control panel 120, the sensors 151, 152, and/or various other components of the PD system 100.

The PD cycler 102 may also include a data storage and/or transmission component 142 that may be coupled to the control unit 140 and that may enable the storage of data on the PD cycler and/or the transmission of data to and from the PD cycler 102. In various implementations, the data may include prescription information, treatment data and/or other therapy-based data and/or may include authorization information and/or other user profile-based information. In various implementations, the component 142 may include a wired connection to a network/Internet, an interface for receiving a physical storage unit, such as a universal serial bus (USB) memory unit, that may be used to transfer and receive data and/or wireless transmission components for transmitting or receiving data and/or other signals wirelessly. The wireless transmission components may include components for short range wireless transmission technologies, such as Bluetooth and/or near-field communications (NFC) technologies, for communication with one or more peripheral devices and/or network transmission component. The communication may include transmitting and receiving data and/or other signals wirelessly via a telecommunications network and/or the Internet to/from one or more remote servers. In connection with transmission, data may be secured and/or encrypted via the control unit 140 using appropriate security and encryption protocols according to applicable laws and regulations governing transmission of sensitive data and/or protected medical information.

In an embodiment, an organizer anchor assembly 160 according to the system described herein may include an organizer unit 161 coupled to an anchor pad or base 162 having a flat bottom with sufficient surface area in contact with a table or other surface to secure the organizer assembly in place during use. The organizer unit 161, such as a stay-safe organizer from Fresenius Medical Care, holds and organizes lines of the PD machine. The anchor base 162 is principally illustrated herein as having a rectangular shape; however, other appropriate shapes may also be used in connection with the system described herein to perform the securing functionality of the organizer anchor assembly 160 to the cart or table. The anchor base 162 may be flat to contact the cart or table surface and/or may include a perpendicular portion designed to wrap around an edge of the cart or table surface. In this way, the organizer anchor assembly 160 may include components that contact one or more surfaces (e.g. front and/or top surfaces) of a cart or table on which the organizer anchor assembly 160 to facilitate stability of the organizer anchor assembly.

The organizer anchor assembly 160 may be beneficially used in connection with a home dialysis machine, such as the PD cycler 102, in which lines and connectors are desirably organized and anchored during a dialysis treatment. As discussed in detail herein, portions of the organizer anchor assembly 160 may be made of one or more materials sufficiently structural to function to securely hold and protect lines in place while providing a frictional interface in contact with a surface to resist movement of the organizer anchor assembly across the surface.

In an embodiment, the anchor base 162 may be made of and/or otherwise include a flexible, rubber-like material, such as silicone or other polymer or elastomer material, and thereby use the material's natural “stickiness” or frictional attachment to provide a fixation to the table or cart. A material such as silicone can attach to uneven surfaces, and since the entire anchor base 162 makes contact, it is more secure than multiple small suction cup feet. Further, silicone can be easily cleaned with soap and water, and cleaning restores the stickiness of the anchor base 162 if it has lost such stickiness after becoming dirty.

The organizer anchor assembly 160 may be conveniently repositioned which makes putting it away easy as well as making it portable for travel. The size of the line holder assembly may small, for example, smaller than the dialysis machine. Further, making the assembly small eliminates the need for right or left handed versions; there may be simply one version that can be placed on the right or left side of the dialysis machine without having to configure specifically the organizer anchor assembly 160. An area of the bottom corner of the silicone pad of the anchor base 162 may be textured and/or otherwise made of a different material to make the area less sticky and provide an area which could be grabbed with fingers and used to help remove it from the table or cart surface when the patient wants to reposition it or travel with it.

One of the risks associated with line holders and organizers is if someone hits or bumps into the assembly, the force could break the line holder or cause pain to the patient. The system described herein includes an organizer anchor assembly 160 with an anchor base 162 that is impact resistant in that it will have natural “give” which would lessen the force upon an impact, thereby reducing the probability of the organizer anchor assembly 160 to move or break.

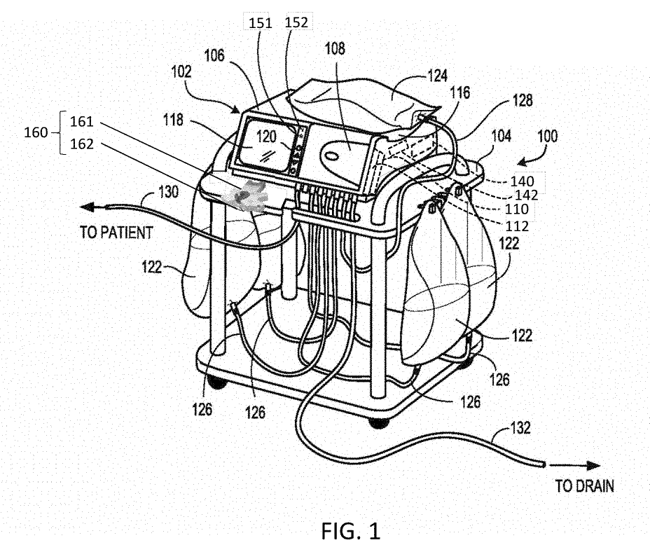

FIG. 2 is a schematic view of the organizer anchor assembly 160 according to an embodiment of the system described herein. The organizer anchor assembly 160 is shown including the organizer unit 161 and the anchor base 162 that would contact a cart or table surface. The anchor base 162 may be made of a polymer material, such as silicone, that restricts movement across the table surface and that is impact resistant. In the figure, the anchor base 162 is shown extended from the organizer unit 161 and having a portion that is perpendicular to the cart or table surface on which the organizer anchor assembly 160 is to be disposed. However, in other embodiments, the anchor base 162 may be an entirely flat portion that does not extend beyond the organizer unit 161 profile.

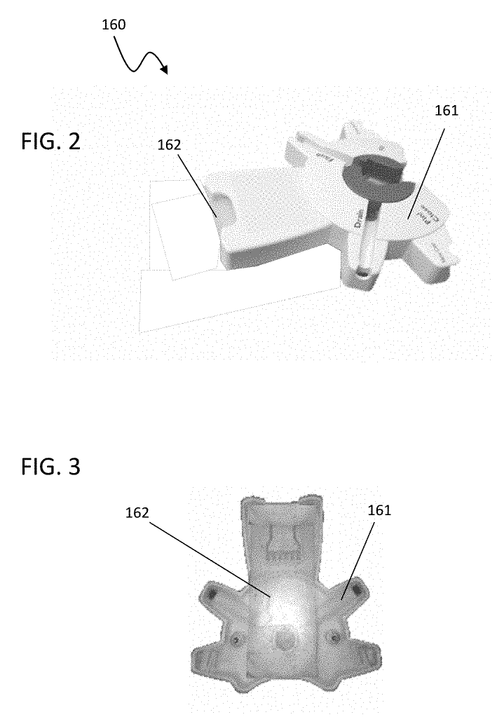

FIG. 3 is a schematic view showing an underside of the organizer anchor assembly 160, specifically the side of an anchor base 162 that would physically contact the cart or table surface.

The anchor base 162 may be made of a polymer material, such as silicone, that resists movement of the organizer anchor assembly 160 across the cart or table surface. In an embodiment, the anchor base 162 may cover at least 50% of the contact surface area of the organizer anchor assembly 160 on the cart or table surface and thereby provide enhanced adhesion or “stickiness” of the organizer anchor assembly 160 to the cart or table surface compared to that of, for example, suction cups. The anchor base 162 may extend along substantially the entire length of the organizer anchor assembly 160.

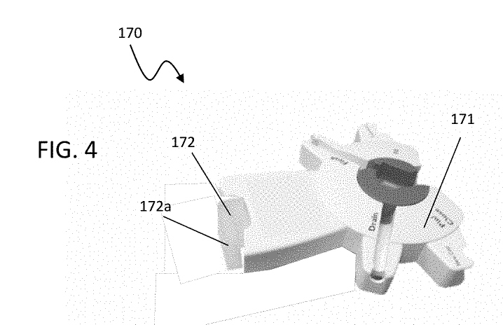

FIG. 4 is a schematic view of an embodiment for an organizer anchor assembly 170, like that discussed herein, in which an anchor base 172 is shown having a portion 172a that extends substantially perpendicular to the underside of an organizer unit 170. In this way, the anchor base 172 may have a portion that wraps around an edge of a cart of table surface to secure the organizer anchor assembly 170 to the cart or table surface.

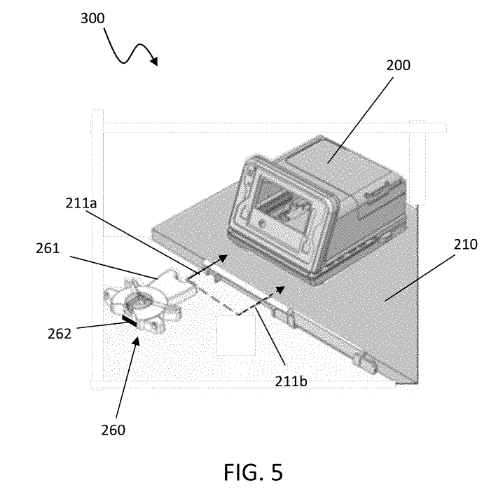

FIG. 5 is a schematic view of a dialysis system 300 including an organizer anchor assembly 260 (e.g. like organizer anchor assembly 160, 170) that may be removably secured to a table surface 210 at a desired location on a table surface 210 and receive lines from a dialysis machine (e.g., PD cycler) 200. The organizer anchor assembly 260 may be used during operation of the dialysis machine 200 when medical fluid is flowing in the lines running to and from the dialysis machine 200, and may be positioned at any appropriate location, such as positions 211a, 211b, on the table surface 210. As further discussed elsewhere herein, the organizer anchor assembly 260 includes an organizer unit 261 coupled to an anchor base 262 that is made of an impact-resistant polymer material, such as silicone, that secures the organizer anchor assembly 260 in place on the table surface 210 to resist movement of the organizer anchor assembly 260 across the table surface and that is resistant to impacts. The system described herein may be advantageously used in connection with any type of dialysis machine, including home dialysis machines, that would benefit from the holding and organizing of lines running to and from the dialysis machine.

Various embodiments discussed herein may be combined with each other in appropriate combinations in connection with the system described herein. Additionally, in some instances, the order of steps in the flow diagrams, flowcharts and/or described flow processing may be modified, where appropriate. Additionally, although the system described herein has been discussed principally in connection with PD cyclers and treatment, in various embodiments the system described herein may also be used in connection with other medical machines and treatments using lines and connectors that are desirably organized and anchored.

Embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. An organizer anchor assembly, comprising:
an organizer unit that is configured to receive and secure a line of a medical device; and
an anchor base coupled to the organizer unit, the anchor base including a contact portion comprising a polymer material that, when in contact with a surface, resists movement of the organizer anchor assembly across the surface.

2. The organizer anchor assembly according to claim 1, wherein the medical device is a dialysis machine.

3. The organizer anchor assembly according to claim 2, wherein the dialysis machine is a peritoneal dialysis machine.

4. The organizer anchor assembly according to claim 1, wherein the contact portion is an impact-resistant polymer material layer of the anchor base.

5. The organizer anchor assembly according to claim 1, wherein the polymer material is silicone.

6. The organizer anchor assembly according to claim 1, wherein the contact portion of the anchor base covers at least 50% of a surface area of a side of the organizer anchor assembly that contacts the surface.

7. The organizer anchor assembly according to claim 1, wherein the contact portion of the anchor base extends substantially along an entire length of the organizer unit.

8. The organizer anchor assembly according to claim 1, wherein the contact portion of the anchor base includes a portion that is substantially perpendicular to a plane of the anchor base that is in contact with the cart or table surface.

9. A medical system, comprising:
a medical device having lines through which medical fluid flows during a medical treatment; and
an organizer anchor assembly, comprising:
an organizer unit that is configured to receive and secure a line of a medical device; and
an anchor base coupled to the organizer unit, the anchor base including a contact portion comprising a polymer material that, when in contact with a surface, resists movement of the organizer anchor assembly across the surface.

10. The medical system according to claim 9, wherein the medical device is a dialysis machine.

11. The medical system according to claim 10, wherein the dialysis machine is a peritoneal dialysis machine.

12. The medical system according to claim 9, wherein the contact portion is an impact-resistant polymer material layer of the anchor base.

13. The medical system according to claim 9, wherein the polymer material is silicone.

14. The medical system according to claim 9, wherein the contact portion of the anchor base covers at least 50% of a surface area of a side of the organizer anchor assembly that contacts the surface.

15. The medical system according to claim 9, wherein the contact portion of the anchor base extends substantially along an entire length of the organizer unit.

16. The medical system according to claim 9, wherein the contact portion of the anchor base includes a portion that is substantially perpendicular to a plane of the anchor base that is in contact with the cart or table surface.
